(19) United States
(12) Patent Application Publication
(10) Pub. No.: US 2025/0099737 A1
(43) Pub. Date: Mar. 27, 2025

(54) ELECTROMAGNETICALLY-DRIVEN HEART PUMP

(71) Applicant: CARDIOFORMA LLC, Jupiter, FL (US)

(72) Inventor: David Esteban PANIAGUA GONZALEZ, Jupiter, FL (US)

(21) Appl. No.: 18/891,378

(22) Filed: Sep. 20, 2024

Related U.S. Application Data
(60) Provisional application No. 63/539,784, filed on Sep. 21, 2023.

(51) Int. Cl. A61M 60/17; A61M 60/232; A61M 60/419; A61M 60/515; A61M 60/90; G16H 40/63

(52) U.S. Cl. CPC A61M 60/17; A61M 60/232; A61M 60/419; A61M 60/515; A61M 60/90; G16H 40/63; A61M 2205/0216; A61M 2205/04; A61M 2205/103; A61M 2205/3303; A61M 2205/3327; A61M 2205/3365; A61M 2205/50; A61M 2205/8206; A61M 2210/125

(57) ABSTRACT

Disclosed herein is a novel implantable transapical expandable heart pump with an extracorporeal motor, battery, and microprocessor that incorporates a valve. The pump can be placed through transapical puncture and does not require cutting the heart. The device can function in the left side of the heart expanding in the left ventricle and extending across the aortic valve to the ascending aorta; as well as the right side of the heart expanding in the right ventricle crossing the pulmonary valve into the main pulmonary artery. The device can replace full heart function when both sides are implanted.

### CROSS-REFERENCE TO RELATED APPLICATIONS

This application claims the benefit of U.S. Provisional Patent Application No. 63/539,784 filed on Sep. 21, 2023 and entitled “A Novel Fully Implantable Transapical Expandable Heart Pump with Extracorporeal Motor and Battery,” which is incorporated by reference herein.

### TECHNICAL FIELD

The present disclosure is directed at least to the fields of surgery, medicine, and medical devices, and more specifically to medical devices for assisting or replacing the heart in blood circulation and methods for implanting and operating such medical devices.

### BACKGROUND

Heart pumps are medical devices that support a failing or failed heart by transferring fluid from one location to another in the human body, such as by transferring fluid from the left ventricle to the aorta or transferring fluid from the right ventricle to the pulmonary artery. Heart pumps may be used to support the patient until a transplant is available or as destination therapy to patients who currently do not have any other option.

Implantation of heart pumps involves a surgical procedure. Implantation of a conventional heart pump involves cutting the apex of the left ventricle to accommodate the pump inflow cannula. The surgical procedure also involves creating an opening in the ascending aorta for connecting the outflow cannula of the pump. Performing this extensive and complex surgical procedure involves use of a cardiopulmonary bypass, which involves risks and potential adverse outcomes. The conventional heart pump requires wires that pierce through the skin for connectivity and power delivery to the pump motor.

### BRIEF SUMMARY

Conventional placement techniques for heart pumps offer at least two significant shortcomings. First, implantation involves use of incisions to gain access to the interior of the heart for heart pump placement. This is a significant intrusion to the heart and can lead to complications and poor patient outcomes. Second, conventional heart pumps require elements, such as wires and cables, to pierce the skin for extracorporeal connectivity. Wires and/or cables piercing the skin in this manner are sources of infection and complications. Furthermore, heart pumps or left ventricular assist devices (LVADs) have a metal inflow tube inserted in the left ventricle and have an outflow cannula that is connected to the aorta by means of extensive surgical procedure that requires placing the patient in a heart and lung machine.

Shortcomings mentioned here are only representative and are included simply to highlight that a need exists for improved heart pumps and surgical procedures. Embodiments described herein address certain shortcomings but not necessarily each and every one described here or known in the art. Furthermore, embodiments described herein may present other benefits than, and be used in other ways than, those of the surgical procedures described above. For example, embodiments of a heart pump according to this disclosure include a geometric housing secured to the outside of a patient that wirelessly powers a geometric plate. The geometric plate which is connected to the proximal end of a cannula is positioned within the body of the patient. The distal end of the cannula is inserted into the heart through a catheter. Once inside the heart, the cannula is expanded and able to move fluid from its position within the heart to another position as needed for effective cardiovascular flow. Such embodiments of a heart pump offer many benefits. For example, such a heart pump may be placed without surgical incisions in the heart tissue and/or without protruding elements piercing the skin. Further, such intervention may be done without the need of placing the patient on a heart and lung machine; thereby, avoiding the complications associated with the use of the heart and lung machine such as stroke, heart attack, aortic dissection, aortic valve insufficiency, or even death.

The present disclosure is directed to a solution to the problems associated with heart pump placement and operations. In some embodiments, the present disclosure may be used in cardiac procedures, including heart pump placement. In some embodiments, the present disclosure will be a transapical expandable heart pump that houses the motor and battery outside of the body and incorporates a self-expanding ventriculo-arterial valvular conduit as well as a cannula valve to replace and reinforce the native valve function. In some embodiments, a heart pump may use centrifugal or axial flow to transfer fluid from one location to another. In some embodiments, a heart pump may make use of an electrical magnetic field to propel an impeller via electromagnetic coupling.

The present disclosure is further directed to systems for inserting a heart pump into the heart of a patient using a novel tissue-piercing placement mechanism. A heart pump system comprises at least one of a geometric housing; at least one of geometric plate coupled to the geometric housing, wherein the geometric housing is outside the body (e.g. extracorporeal) of the patient and the geometric plate is positioned within the body of the patient, wherein a barrier of skin is positioned between the geometric housing and the geometric plate; at least one cannula element coupled to the geometric plate, wherein the at least one cannula is configured to penetrate at least one chamber of a heart, wherein the at least one cannula is configured to eject blood from within the at least one chamber of the heart to outside the at least one chamber of the heart. The heart pump system and its various elements may be replaced or removed.

In various embodiments, the cannula comprises a frame with a membrane wherein the frame is coated with a pharmaceutical drug such as but not limited to drug eluting anticoagulants. The cannula may be positioned inside a delivery sheath. The delivery sheath may be coupled to an introducer tip. The cannula may comprise a self-expanding cannula. After placement, the self-expanding cannula may transition from a first formation into a second formation. For example, one element of the cannula, such as the frame, may transition from a compressed position to an expanded position after the sheath has been removed. In the expanded position, the frame may make contact with a cardiac valve. The cannula comprises at least one of a dual cannula or an axial cannula. The dual cannula comprises at least one of a frame, an outflow conduit, an inflow conduit, an impeller housing, a one or more valves, a one or more sensors, or an impeller housing ventricle interface. The axial cannula comprises at least one of a frame, an outflow conduit, an impeller, an axial shaft, a connector that couples the at least one axial shaft to at least one flex drive shaft; one or more valves, a one or more sensors. The impeller housing comprises at least an impeller. The impeller comprises at least one of a centrifugal impeller or an axial impeller. The geometric housing comprises at least one of a motor, a battery, a microprocessors, a chest frame attachment mechanism, a geometric motor interface or a body plate motor connector. The geometric plate comprises at least one of a ventricular magnetic driver housing, a ventricle magnetic impeller driver connector, or a ventricle magnetic impeller interface. The geometric housing may have a configuration comprising one or more of a triangle, a circle, orthogonal, hexagonal, or a different geometry. The dual cannula is coupled to the geometric plate by a flex drive wherein the flex drive can be flexed to accommodate a different anatomical configuration of the patient. The chest frame attachment mechanism includes a one or more magnets powered by electricity, or mechanical attachment mechanism to hold the geometric housing in place.

In some examples, the introducer tip comprises a needle, catheter, dilator, trocar, etc. The introducer tip comprises a catheter and/or needle, such as but not limited to as micro puncture needle or Cook needle, having size 6 G, 7 G, 8 G, 9 G, 10 G, 11 G, 12 G, 13 G, 14 G, 15 G, 16 G, 17 G, 18 G, 19 G, 20 G, 21 G, 22 G, 23 G, 24 G, 25 G, 26 G, 27 G, 28 G, 29 G, 30 G, 31 G, 32 G, 33 G, 34 G, and any range therebetween; where ‘G’ refers to gauge as a measure of wire diameter. The cannula comprises a needle and/or catheter having a length of 0.1 in, 1 in, 2 in, 3 in, 4 in, 5 in, 6 in and any range therebetween. The cannula comprises a trocar having a size of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm and any range therebetween. The cannula comprises a dilator having a length of 0.1 in, 1 in, 2 in, 3 in, 4 in, 5 in, 6 in, 7 in, 8 in, 9 in, 10 in, 11 in, 12 in, 13 in, 14 in, 15 in, 16 in, 17 in and any range therebetween.

In various examples, the frame may have a wall thickness of 0.01 mm to 10 mm. The frame comprises a wall thickness having a size of 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, and any range therebetween. The operational diameter of the frame may range from 0.1 mm to 35 mm, preferably 11 mm to 22 mm. The frame comprises an operational diameter of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, and any range therebetween. The frame may be compressed into a smaller diameter. The compressed diameter may range from 0.1 to 25 mm, preferably 8 mm. The frame comprises a compressed diameter of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, and any range therebetween.

In some embodiments, the axial conduit, the dual canula outflow conduit and/or inflow conduit may have a wall thickness of ranging from 0.01 mm to 2 mm preferably 0.08-0.20 mm. The axial conduit, the dual canula outflow conduit and/or inflow conduit wall thickness comprises 0.01 mm, 0.02 mm, 0.03 mm, 0.04 mm, 0.05 mm, 0.06 mm, 0.07 mm, 0.08 mm, 0.09 mm, 0.10 mm, 0.11 mm, 0.12 mm, 0.13 mm, 0.14 mm, 0.15 mm, 0.16 mm, 0.17 mm, 0.18 mm, 0.19 mm, 0.20 mm, 0.21 mm, 0.22 mm, 0.23 mm, 0.24 mm, 0.25 mm, 0.26 mm, 0.27 mm, 0.28 mm, 0.29 mm, 0.30 mm, 0.31 mm, 0.32 mm, 0.33 mm, 0.34 mm, 0.35 mm, 0.36 mm, 0.37 mm, 0.38 mm, 0.39 mm, 0.40 mm, 0.41 mm, 0.42 mm, 0.43 mm, 0.44 mm, 0.45 mm, 0.46 mm, 0.47 mm, 0.48 mm, 0.49 mm, 0.50 mm, 0.51 mm, 0.52 mm, 0.53 mm, 0.54 mm, 0.55 mm, 0.56 mm, 0.57 mm, 0.58 mm, 0.59 mm, 0.60 mm, 0.61 mm, 0.62 mm, 0.63 mm, 0.64 mm, 0.65 mm, 0.66 mm, 0.67 mm, 0.68 mm, 0.69 mm, 0.70 mm, 0.71 mm, 0.72 mm, 0.73 mm, 0.74 mm, 0.75 mm, 0.76 mm, 0.77 mm, 0.78 mm, 0.79 mm, 0.80 mm, 0.81 mm, 0.82 mm, 0.83 mm, 0.84 mm, 0.85 mm, 0.90 mm, 0.91 mm, 0.92 mm, 0.93 mm, 0.94 mm, 0.95 mm, 0.96 mm, 0.97 mm, 0.98 mm, 0.99 mm, 1 mm, 1.01 mm, 1.02 mm, 1.03 mm, 1.04 mm, 1.05 mm, 1.06 mm, 1.07 mm, 1.08 mm, 1.09 mm, 1.10 mm, 1.11 mm, 1.12 mm, 1.13 mm, 1.14 mm, 1.15 mm, 1.16 mm, 1.17 mm, 1.18 mm, 1.19 mm, 1.20 mm, 1.21 mm, 1.22 mm, 1.23 mm, 1.24 mm, 1.25 mm, 1.26 mm, 1.27 mm, 1.28 mm, 1.29 mm, 1.30 mm, 1.31 mm, 1.32 mm, 1.33 mm, 1.34 mm, 1.35 mm, 1.36 mm, 1.37 mm, 1.38 mm, 1.39 mm, 1.40 mm, 1.41 mm, 1.42 mm, 1.43 mm, 1.44 mm, 0.45 mm, 1.46 mm, 1.47 mm, 1.48 mm, 1.49 mm, 1.50 mm, 1.51 mm, 1.52 mm, 1.53 mm, 1.54 mm, 1.55 mm, 1.56 mm, 1.57 mm, 1.58 mm, 1.59 mm, 1.60 mm, 1.61 mm, 1.62 mm, 1.63 mm, 1.64 mm, 1.65 mm, 1.66 mm, 1.67 mm, 1.68 mm, 1.69 mm, 1.70 mm, 1.71 mm, 1.72 mm, 1.73 mm, 1.74 mm, 1.75 mm, 1.76 mm, 1.77 mm, 1.78 mm, 1.79 mm, 1.80 mm, 1.81 mm, 1.82 mm, 1.83 mm, 1.84 mm, 1.85 mm, 1.90 mm, 1.91 mm, 1.92 mm, 1.93 mm, 1.94 mm, 1.95 mm, 1.96 mm, 1.97 mm, 1.98 mm, 1.99 mm, 2 mm any range therebetween.

In some examples, the physiological measurements my include cannula sensors that measures a one or more of pressure within the chamber of the heart, force of the pump, a blood pressure, a blood oxygenation level, a blood pH level, a blood lactate level, a troponin level, a blood carbon dioxide level, a partial pressure of oxygen, a partial pressure of carbon dioxide, a blood bicarbonate level, a hemoglobin level, a blood sodium level, a blood magnesium level, a blood potassium level, a blood calcium level, a blood chloride level, a blood phosphate level, a blood sugar level, blood procalcitonin level, or blood B-type natriuretic peptide level. The physiological measurements may be used by a healthcare professional and/or an artificial intelligence algorithm to determine a historical value based on prior physiological measurement values. A healthcare professional and/or an artificial intelligence algorithm may then determine a threshold based on the historical value. Additionally, the healthcare professional and/or artificial intelligence algorithm may adjust the speed of the motor based on a comparison between the physiological measurement value and the threshold.

For one example, the geometric housing comprises one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In some embodiments, the geometric plate may comprise one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In one example, the cannula plate may comprise one or more polymers, one or more metals, etc., one or more ceramics or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In other embodiments, the frame may comprise one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In some embodiments, the impeller and/or the impeller housing may be compromised of one or more biologically inert material polymers, one or more metals, one or more ceramic, etc. or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof. The impeller housing may be manufactured using additive manufacturing such 3D printing.

In some embodiments, the geometric motor interface to the body plate motor connector and ventricle magnetic impeller driver interface may be manufactured of polymers, metals, ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. In some embodiments, the ceramics may be comprised of alumina, zirconia, calcium orthophosphates, bioactive glasses, silicon carbide, or a combination thereof.

In one embodiment, the sheath may be composed of a one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In one example, the frame may comprise a pharmaceutical compound coating comprising of one or more of heparin, warfarin, apixaban, dabigatran, rivaroxaban, enoxaparin, thrombin inhibitors, edoxaban, fondaparinux, aspirin, ticagrelor, tirofiban, vorapaxar, clopidogrel, sirolimus, everolimus, zotarolimus, paclitaxel, biolimus, messenger RNA, or a combination thereof.

In some examples, the dual cannula may comprise at least one impeller housing ventricle interface; at least one impeller housing coupled to the impeller housing ventricle interface; at least one inflow conduit coupled to the impeller housing ventricle interface; an outflow conduit with a proximal end and a distal end, where in the proximal end is coupled to the inflow conduit; at least one valve coupled to the distal end of the outflow conduit.

In some embodiments, the axial cannula may comprise at least one shield; at least one axial impeller positioned within the shield; at least one cannula shaft coupled to the axial impeller; at least one connector that couples the at least one axial shaft to at least one flex drive shaft; at least one conduit with a proximal end and a distal end, wherein the proximal end of the conduit is coupled to the axial impeller, wherein the distal end of the conduit is coupled to a one or more valve.

In one embodiment, the outflow conduit, inflow conduit, and the one or more valves may compromise one or more polymers, one or more biological materials, or a combination of one or more polymer and one or more biological material. The polymer may be comprised of polyetheretherketone (PEEK), Polytetrafluoroethylene (PTFE) and polyurethane (PU), ultra-high molecular weight polyethylene, polystyrene, polycarbonates, polypropylene, polyethylene, polyvinylchloride, acrylonitrile butadiene styrene, polystyrene, polyethylene terephthalate glycol, polymethyl methacrylate, or a combination thereof. The biological material may comprise of mammal pericardium such as bovine pericardium, ovine pericardium, porcine pericardium, horse pericardium, kangaroo pericardium, or other biological materials such as mammal placenta, fish cornea, or a combination thereof.

In one example, the motor assembly may comprise: at least one ventricular impeller driver connector; at least one ventricle magnetic driver housing coupled to the at least one ventricular impeller driver connector; at least one motor interface plate coupled to the at least one ventricle magnetic driver housing; at least one housing element coupled to the motor interface plate, wherein skin runs between the at least one housing element and the motor interface plate; wherein the housing element comprises at least one of an extracorporeal motor, a microprocessor, a battery, a chest frame attachment mechanism, or motor magnets; wherein the ventricular magnetic driver housing comprises at least one of impeller driver magnet or ventricular driver; wherein the motor magnets are coupled without contact to the impeller driver magnet.

In some embodiments, the heart pump receives, from a first sensor of an inflow conduit, a cardiovascular pressure reading; receives, from a one or more second sensors of an inflow conduit, a cardiovascular context, wherein the cardiovascular context comprises at least one of the blood pressure level, the partial pressure of oxygen, the partial pressure of carbon dioxide, the lactate level, the blood sugar level, the blood pH level, or any combination thereof; determines, by the processor, a minimum cardiovascular pressure reading based on the cardiovascular context; determines, by the processor, if the cardiovascular pressure reading is below the minimum threshold; adjusting, by the processor, a pump ejection force of an outflow conduit, wherein the cardiovascular pressure reading is below the minimum threshold.

In various embodiments, a flex drive may comprise: at least one of a chest magnetic housing; at least one of a shaft housing element with a proximate end and a distal end, wherein the shaft housing element is capable of flexing in all directions, and wherein the proximate end of the flex housing element is coupled to the at least one chest magnetic housing; at least one ventricle magnetic driver housing coupled to the distal end of the flex housing element.

For example, the present disclosure is also directed to methods for inserting a heart pump into the heart of a patient using a novel tissue-piercing placement mechanism. Aspects of the method may be combined with aspects of the system disclosed above, such as when the method for inserting a heart pump is applied for inserting the heart pump according to embodiments of this disclosure. A method for placing the heart pump in an individual comprising: inserting a cannula into a chamber of the heart through one or more punctures of the heart of the patient; connecting the cannula to a geometric plate; coupling the geometric plate to a geometric housing.

In some embodiments, the heart pump receives, from a first sensor of an inflow conduit, a cardiovascular pressure reading; receives, from a one or more second sensors of an inflow conduit, a cardiovascular context, wherein the cardiovascular context comprises at least one of the blood pressure level, the partial pressure of oxygen, the partial pressure of carbon dioxide, the lactate level, the blood sugar level, the blood pH level, or any combination thereof; determines, by the processor, a minimum cardiovascular pressure reading based on the cardiovascular context; determines, by the processor, if the cardiovascular pressure reading is below the minimum threshold; adjusting, by the processor, a pump ejection force of an outflow conduit, wherein the cardiovascular pressure reading is below the minimum threshold.

In some embodiments, the method comprises one or more steps of: utilizing surgical means to expose the ribs thereby gaining access to the heart. Once heart access is obtained, the health care professional would begin by inserting the needle through the tissue of the heart providing access to the chamber of the heart. A guidewire is passed through the needle and advanced such that the distal end of the guidewire passes through the target cardiac valve. The needle removed from the heart tissue by passing it proximally over the guidewire. The correct placement of the guidewire and removal of the needle is confirmed via anatomical ultrasound techniques and radiological guidance. A sequence of dilators advanced over the wire may precede sheath insertion that is advanced over the guidewire. Once the sheath crosses the target cardiac valve, the unsheathing begins allowing the frame to expand making contact with the target cardiac valve. A shield making contact with outside portion of a target heart chamber is employed. A heart mesh is sutured on to the heart apex. The base of the cannula is coupled to the geometric plate. The geometric housing is positioned extracorporeally such that it may provide wireless power and direction through the to the geometric plate to the cannula.

In some examples, a left ventricle insertion method comprises one or more steps of: utilizing surgical means to expose the ribs thereby gaining access to the heart. Once heart access is obtained, the health care professional would begin by inserting the needle through the tissue of the heart providing access to the left ventricle of the heart. A guidewire is passed through the needle and advanced such that the distal end of the guidewire passes through the aortic valve. The needle removed from the heart tissue by passing it proximally over the guidewire. The correct placement of the guidewire and removal of the needle is confirmed via anatomical ultrasound techniques and radiological guidance. A sheath is advanced across the guidewire. Once the sheath crosses the aortic valve, the unsheathing begins allowing the frame to expand making contact with the aortic valve. A shield making contact with outside portion of a left ventricle is employed. A heart mesh is sutured on to the heart apex. The base of the cannula is coupled to the geometric plate. The geometric housing is positioned extracorporeally such that it may provide wireless power and direction through the to the geometric plate to the cannula.

In some embodiments, a right ventricle insertion method comprises one or more steps of: utilizing surgical means to expose the ribs thereby gaining access to the heart. Once heart access is obtained, the health care professional would begin by inserting the needle through the tissue of the heart providing access to the right ventricle of the heart. A guidewire is passed through the needle and advanced such that the distal end of the guidewire passes through the pulmonic valve. The needle removed from the heart tissue by passing it proximally over the guidewire. The correct placement of the guidewire and removal of the needle is confirmed via anatomical ultrasound techniques and radiological guidance. Once sequential dilators are advanced over the wire, then the sheath is advanced over the guidewire. Once the sheath crosses the pulmonic valve, the unsheathing begins allowing the frame to expand making contact with the aortic valve. A shield making contact with outside portion of a right ventricle is employed. A heart mesh is sutured on to the heart apex. The base of the cannula is coupled to the geometric plate. The geometric housing is positioned extracorporeally such that it may provide wireless power and direction through the to the geometric plate to the cannula.

In one example, a right atrium insertion method comprises one or more steps of: utilizing surgical means to expose the ribs thereby gaining access to the heart. Once heart access is obtained, the health care professional would begin by inserting the needle through the tissue of the heart providing access to the right atrium of the heart. A guidewire is passed through the needle and advanced such that the distal end of the guidewire passes through the right atrioventricular (AV) valve. The needle removed from the heart tissue by passing it proximally over the guidewire. The correct placement of the guidewire and removal of the needle is confirmed via anatomical ultrasound techniques and radiological guidance. A sheath is advanced across the guidewire. Once the sheath crosses the right AV valve, the unsheathing begins allowing the frame to expand making contact with the right AV valve. A shield making contact with outside portion of a right atrium is employed. A heart mesh is then sutured on to the heart. The base of the cannula is coupled to the geometric plate. The geometric housing is positioned extracorporeally such that it may provide wireless power and direction through the to the geometric plate to the cannula.

In some embodiments, a left atrium insertion method comprises one or more steps of: utilizing surgical means to expose the ribs thereby gaining access to the heart. Once heart access is obtained, the health care professional would begin by inserting the needle through the tissue of the heart providing access to the left atrium of the heart. A guidewire is passed through the needle and advanced such that the distal end of the guidewire passes through the left atrioventricular (AV) valve. The needle removed from the heart tissue by passing it proximally over the guidewire. The correct placement of the guidewire and removal of the needle is confirmed via anatomical ultrasound techniques and radiological guidance. A sheath is advanced across the guidewire. Once the sheath crosses the left AV valve, the unsheathing allows the frame to expand making contact with the left AV valve. A shield may make contact with an outside portion of a left AV, and a heart mesh may be sutured on to the heart. The base of the cannula is coupled to the geometric plate. The geometric housing may be positioned extracorporeally such that it may provide wireless power and direction through the to the geometric plate to the cannula.

In various embodiments, the cannula comprises a frame with a membrane wherein the frame is coated with a pharmaceutical drug such as but not limited to drug eluting anticoagulants. The cannula may be positioned inside a delivery sheath. The delivery sheath may be coupled to an introducer tip. The cannula comprises at least one of a dual cannula or an axial cannula. The dual cannula comprises at least one of a frame, an outflow conduit, an inflow conduit, an impeller housing, a one or more valves, a one or more sensors, or an impeller housing ventricle interface. The axial cannula comprises at least one of a frame, an outflow conduit, an impeller, an axial shaft, a connector that couples the at least one axial shaft to at least one flex drive shaft; one or more valves, a one or more sensors. The impeller housing comprises at least an impeller. The impeller comprises at least one of a centrifugal impeller or an axial impeller. The geometric housing comprises at least one of a motor, a battery, a microprocessors, a chest frame attachment mechanism, a geometric motor interface or a body plate motor connector. The geometric plate comprises at least one of a ventricular magnetic driver housing, a ventricle magnetic impeller driver connector, or a ventricle magnetic impeller interface. The geometric housing may have a configuration comprising one or more of a triangle, a circle, orthogonal, hexagonal, or a different geometry. The dual cannula is coupled to the geometric plate by a flex drive wherein the flex drive can be flexed to accommodate a different anatomical configuration of the patient. The chest frame attachment mechanism includes a one or more magnets powered by electricity, or mechanical attachment mechanism to hold the geometric housing in place.

In some examples, the introducer tip comprises a needle, catheter, dilator, trocar, etc. The introducer tip comprises a catheter and/or needle, such as but not limited to micro puncture needle or Cook needle, having size 6 G, 7 G, 8 G, 9 G, 10 G, 11 G, 12 G, 13 G, 14 G, 15 G, 16 G, 17 G, 18 G, 19 G, 20 G, 21 G, 22 G, 23 G, 24 G, 25 G, 26 G, 27 G, 28 G, 29 G, 30 G, 31 G, 32 G, 33 G, 34 G, and any range therebetween. The cannula comprises a needle and/or catheter having a length of 0.1 in, 1 in, 2 in, 3 in, 4 in, 5 in, 6 in and any range therebetween. The cannula comprises a dilator or a trocar having a size of 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm and any range therebetween. The cannula comprises a dilator or trocar having length of 0.1 in, 1 in, 2 in, 3 in, 4 in, 5 in, 6 in, 7 in, 8 in, 9 in, 10 in, 11 in, 12 in, 13 in, 14 in, 15 in, 16 in, 17 in and any range therebetween.

For example, the frame may have a wall thickness of 0.01 mm to 10 mm. The frame comprises a wall thickness having a size of 0.01 mm, 0.02 mm, 0.03 mm, 0.04 mm, 0.05 mm, 0.06 mm, 0.07 mm, 0.08 mm, 0.09 mm, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, and any range therebetween. The operational diameter of the frame may range from 0.1 mm to 35 mm, preferably 11 mm to 22 mm. The frame comprises an operational diameter of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, and any range therebetween. The frame may be compressed into a smaller diameter. The compressed diameter may range from 0.1 to 25 mm, preferably 8 mm. The frame comprises a compressed diameter of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, and any range therebetween.

In some embodiments, the axial conduit, the dual canula outflow conduit and/or inflow conduit may have a wall thickness of ranging from 0.01 mm to 2 mm preferably 0.08-0.20 mm. The axial conduit, the dual canula outflow conduit and/or inflow conduit wall thickness comprises 0.01 mm, 0.02 mm, 0.03 mm, 0.04 mm, 0.05 mm, 0.06 mm, 0.07 mm, 0.08 mm, 0.09 mm, 1.0 mm, 0.11 mm, 0.12 mm, 0.13 mm, 0.14 mm, 0.15 mm, 0.16 mm, 0.17 mm, 0.18 mm, 0.19 mm, 0.20 mm, 0.21 mm, 0.22 mm, 0.23 mm, 0.24 mm, 0.25 mm, 0.26 mm, 0.27 mm, 0.28 mm, 0.29 mm, 0.30 mm, 0.31 mm, 0.32 mm, 0.33 mm, 0.34 mm, 0.35 mm, 0.36 mm, 0.37 mm, 0.38 mm, 0.39 mm, 0.40 mm, 0.41 mm, 0.42 mm, 0.43 mm, 0.44 mm, 0.45 mm, 0.46 mm, 0.47 mm, 0.48 mm, 0.49 mm, 0.50 mm, 0.51 mm, 0.52 mm, 0.53 mm, 0.54 mm, 0.55 mm, 0.56 mm, 0.57 mm, 0.58 mm, 0.59 mm, 0.60 mm, 0.61 mm, 0.62 mm, 0.63 mm, 0.64 mm, 0.65 mm, 0.66 mm, 0.67 mm, 0.68 mm, 0.69 mm, 0.70 mm, 0.71 mm, 0.72 mm, 0.73 mm, 0.74 mm, 0.75 mm, 0.76 mm, 0.77 mm, 0.78 mm, 0.79 mm, 0.80 mm, 0.81 mm, 0.82 mm, 0.83 mm, 0.84 mm, 0.85 mm, 0.90 mm, 0.91 mm, 0.92 mm, 0.93 mm, 0.94 mm, 0.95 mm, 0.96 mm, 0.97 mm, 0.98 mm, 0.99 mm, 1 mm, 1.01 mm, 1.02 mm, 1.03 mm, 1.04 mm, 1.05 mm, 1.06 mm, 1.07 mm, 1.08 mm, 1.09 mm, 1.10 mm, 1.11 mm, 1.12 mm, 1.13 mm, 1.14 mm, 1.15 mm, 1.16 mm, 1.17 mm, 1.18 mm, 1.19 mm, 1.20 mm, 1.21 mm, 1.22 mm, 1.23 mm, 1.24 mm, 1.25 mm, 1.26 mm, 1.27 mm, 1.28 mm, 1.29 mm, 1.30 mm, 1.31 mm, 1.32 mm, 1.33 mm, 1.34 mm, 1.35 mm, 1.36 mm, 1.37 mm, 1.38 mm, 1.39 mm, 1.40 mm, 1.41 mm, 1.42 mm, 1.43 mm, 1.44 mm, 0.45 mm, 1.46 mm, 1.47 mm, 1.48 mm, 1.49 mm, 1.50 mm, 1.51 mm, 1.52 mm, 1.53 mm, 1.54 mm, 1.55 mm, 1.56 mm, 1.57 mm, 1.58 mm, 1.59 mm, 1.60 mm, 1.61 mm, 1.62 mm, 1.63 mm, 1.64 mm, 1.65 mm, 1.66 mm, 1.67 mm, 1.68 mm, 1.69 mm, 1.70 mm, 1.71 mm, 1.72 mm, 1.73 mm, 1.74 mm, 1.75 mm, 1.76 mm, 1.77 mm, 1.78 mm, 1.79 mm, 1.80 mm, 1.81 mm, 1.82 mm, 1.83 mm, 1.84 mm, 1.85 mm, 1.90 mm, 1.91 mm, 1.92 mm, 1.93 mm, 1.94 mm, 1.95 mm, 1.96 mm, 1.97 mm, 1.98 mm, 1.99 mm, 2 mm any range therebetween.

In one example, wherein physiological measurements may include cannula sensors that measures a one or more of pressure within the chamber of the heart, force of the pump, a blood pressure, a blood oxygenation level, a blood pH level, a blood lactate level, a troponin level, a blood carbon dioxide level, a partial pressure of oxygen, a partial pressure of carbon dioxide, a blood bicarbonate level, a hemoglobin level, a blood sodium level, a blood magnesium level, a blood potassium level, a blood calcium level, a blood chloride level, a blood phosphate level, a blood sugar level, blood procalcitonin level, or blood B-type natriuretic peptide level. The physiological measurements may be used by a healthcare professional and/or an artificial intelligence algorithm to determine a historical value based on prior physiological measurement values. A healthcare professional and/or an artificial intelligence algorithm may then determine a threshold based on the historical value. Additionally, the healthcare professional and/or artificial intelligence algorithm may adjust the speed of the motor based on a comparison between the physiological measurement value and the threshold.

In some examples, the geometric housing may comprise one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

For example, the geometric plate may comprise one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In one embodiment, the cannula plate may comprise one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In one example, the frame may comprise one or more polymers, one or more metals, one or more ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In some embodiments, the impeller and/or the impeller housing may be compromised of one or more biologically inert material polymers, one or more metals, one or more ceramic, etc. or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof. The impeller housing may be manufactured using additive manufacturing such 3D printing.

In some embodiments, the geometric motor interface to the body plate motor connector and ventricle magnetic impeller driver interface may be manufactured of polymers, metals, ceramics, etc., or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. In some embodiments, the ceramics may be comprised of alumina, zirconia, calcium orthophosphates, bioactive glasses, silicon carbide, or a combination thereof.

For example, the sheath may be composed of a one or more polymers, one or more metals, one or more ceramics, etc. or a combination thereof. The polymer may be comprised of Polytetrafluoroethylene (PTFE) and polyurethane (PU), ultra-high molecular weight polyethylene, polyetheretherketone (PEEK), polystyrene, polycarbonates, polypropylene, polyethylene, polyvinylchloride, acrylonitrile butadiene styrene, polystyrene, polyethylene terephthalate glycol, polymethyl methacrylate, or a combination thereof. The metals may be comprised of stainless steel, nitinol, aluminum, copper, titanium, cobalt chrome, magnesium, gold, platinum, silver, iridium, tantalum, or a combination thereof. The ceramics may be comprised of alumina, zirconia, calcium orthophosphates, bioactive glasses, silicon carbide, or a combination thereof.

In one example, the frame may comprise a pharmaceutical compound coating comprising of one or more of heparin, warfarin, apixaban, dabigatran, rivaroxaban, enoxaparin, thrombin inhibitors, edoxaban, fondaparinux, aspirin, ticagrelor, tirofiban, vorapaxar, clopidogrel, sirolimus, everolimus, zotarolimus, paclitaxel, biolimus, messenger RNA, or a combination thereof.

In one embodiment, the dual cannula may comprise: at least one impeller housing ventricle interface; at least one impeller housing coupled to the impeller housing ventricle interface; at least one inflow conduit coupled to the impeller housing ventricle interface; an outflow conduit with a proximal end and a distal end, where in the proximal end is coupled to the inflow conduit; at least one valve coupled to the distal end of the outflow conduit.

In some embodiments, the axial cannula may comprise at least one shield; at least one axial impeller positioned within the shield; at least one cannula shaft coupled to the axial impeller; at least one connector that couples the at least one axial shaft to at least one flex drive shaft; at least one conduit with a proximal end and a distal end, wherein the proximal end of the conduit is coupled to the axial impeller, wherein the distal end of the conduit is coupled to a one or more valve.

For example, the outflow conduit, inflow conduit, and the one or more valves may compromise one or more polymers or one or more biological materials or a combination of one or more polymer and one or more biological material. The polymer may be comprised of polyetheretherketone (PEEK), Polytetrafluoroethylene (PTFE) and polyurethane (PU), ultra-high molecular weight polyethylene, polystyrene, polycarbonates, polypropylene, polyethylene, polyvinylchloride, acrylonitrile butadiene styrene, polystyrene, polyethylene terephthalate glycol, polymethyl methacrylate, or a combination thereof. The biological material may comprise of mammal pericardium such as bovine pericardium, ovine pericardium, porcine pericardium, horse pericardium, kangaroo pericardium, mammal placenta, fish cornea, or a combination thereof.

In one example, the motor assembly may comprise: at least one ventricular impeller driver connector; at least one ventricle magnetic driver housing coupled to the at least one ventricular impeller driver connector; at least one motor interface plate coupled to the at least one ventricle magnetic driver housing; at least one housing element coupled to the motor interface plate, wherein skin runs between the at least one housing element and the motor interface plate; wherein the housing element comprises at least one of an extracorporeal motor, a microprocessor, a battery, a chest frame attachment mechanism, or motor magnets; wherein the ventricular magnetic driver housing comprises at least one of impeller driver magnet or ventricular driver; wherein the motor magnets are coupled without contact to the impeller driver magnet.

In some embodiments, the heart pump receives, from a first sensor of an inflow conduit, a cardiovascular pressure reading; receives, from a one or more second sensors of an inflow conduit, a cardiovascular context, wherein the cardiovascular context comprises at least one of the blood pressure level, the partial pressure of oxygen, the partial pressure of carbon dioxide, the lactate level, the blood sugar level, the blood pH level, or any combination thereof; determines, by the processor, a minimum cardiovascular pressure reading based on the cardiovascular context; determines, by the processor, if the cardiovascular pressure reading is below the minimum threshold; adjusting, by the processor, a pump ejection force of an outflow conduit, wherein the cardiovascular pressure reading is below the minimum threshold.

In some embodiments, a flex drive may comprise: at least one of a chest magnetic housing; at least one of a shaft housing element with a proximate end and a distal end, wherein the shaft housing element is capable of flexing in all directions, and wherein the proximate end of the flex housing element is coupled to the at least one chest magnetic housing; at least one ventricle magnetic driver housing coupled to the distal end of the flex housing element.

In some embodiments, the heart pump receives, from a first sensor of an inflow conduit, a cardiovascular pressure reading; receives, from a one or more second sensors of an inflow conduit, a cardiovascular context, wherein the cardiovascular context comprises at least one of the blood pressure level, the partial pressure of oxygen, the partial pressure of carbon dioxide, the lactate level, the blood sugar level, the blood pH level, or any combination thereof; determines, by the processor, a minimum cardiovascular pressure reading based on the cardiovascular context; determines, by the processor, if the cardiovascular pressure reading is below the minimum threshold; adjusting, by the processor, a pump ejection force of an outflow conduit, wherein the cardiovascular pressure reading is below the minimum threshold.

### BRIEF DESCRIPTION OF THE DRAWINGS

For a more complete understanding of the present disclosure, reference is now made to the following descriptions taken in conjunction with the accompanying drawing, in which.

The present embodiments now will be discussed in detail with an emphasis on highlighting the advantageous features. These embodiments are for illustrative purposes only and are not to scale, instead emphasizing the principles of the disclosure. These drawings include the following figures, in which like numerals may indicate like parts:

FIG. 1A illustrates an example embodiment of the heart pump placement in the human body, and the extracorporeal motor and battery outside the human body.

FIG. 1B illustrates an isometric view of the device according to some embodiments of the disclosure.

FIG. 2A illustrates a side view of the motor and battery according to some embodiments of the disclosure outside the human body.

FIG. 2B illustrates a side view of the motor and battery according to some embodiments of the disclosure after connection outside of the human body.

FIG. 3A illustrates a side view of the heart pump according to some embodiments of the disclosure.

FIG. 3B illustrates a top view of the heart pump according to some embodiments of the disclosure.

FIG. 3C illustrates a segmented view of FIG. 3B between section A-A.

FIG. 4A shows a side view of the motor and battery outside the human body with flex drive according to some embodiments of the disclosure.

FIG. 4B shows a side view of the motor and battery being connected to the outside of the human body for the centrifugal heart pump according to some embodiments of the disclosure.

FIG. 5A-B shows an isometric and exploded view of one embodiment of the disclosure.

FIG. 5C shows a side view of the flex drive according to some embodiments of the disclosure.

FIG. 6A shows a side view of an example device with a flex drive.

FIG. 6B shows a side view of FIG. 6A segmented between section A-A.

FIG. 7A-F shows an example of the insertion method for a heart pump according to some embodiments of the disclosure.

FIG. 8A shows a top view of a dual cannula according to some embodiments of the disclosure.

FIG. 8B shows a partial side view from FIG. 8A segmented between section F-F.

FIG. 8C shows a partial side view from FIG. 8A segmented between section F-F, illustrating the inflow and outflow as well as the flow profile inside the dual cannula 243 after removing the frame.

FIG. 8D shows an isometric view from FIG. 8A segmented between section F-F, illustrating the inflow and outflow flow profile of the device removing the frame and outflow conduit.

FIG. 8E shows a side view of the impeller housing according to some embodiments of the disclosure, with dash lines illustrating the inner cavity.

FIG. 8F shows side view of the impeller housing according to some embodiments of the disclosure, and three segmented views: BB-BB, BC-BC, and BD-BD illustrating the inner cavity.

FIG. 9A shows a side view of an axial heart pump according to some embodiments of the disclosure.

FIG. 9B shows a side view from FIG. 9A segmented between section A-A of the axial heart pump.

FIG. 9C shows a top view of an example of an axial heart pump.

FIG. 10 shows a flow profile indicating inflow and outflow of the axial pump according to some embodiments of the disclosure.

FIG. 11A-B shows a side view of the motor and battery connection for a heart pump with the axial cannula for the circular configuration and the geometric configuration connected to the outside of the human body according to some embodiments of the disclosure.

FIG. 11C illustrates the placement of a heart pump according to some embodiments of the disclosure with the axial cannula in the human heart.

FIG. 12 illustrates an example of the wireless charging and wireless connectivity of the heart pump according to some embodiments of the disclosure.

FIG. 13 is a block diagram illustrating a power transmitting network that employs resonant magnetic coupling, ultrasonic frequency, and/or radio frequency for wireless power transfer and communication from a power source to the heart pump system according to some embodiments of the disclosure.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

As used herein, the terms “proximal” and “distal” are used with reference to an operator (e.g., doctor, nurse, technician) such that a proximal side refers to the side closer to the operator and a distal side refers to the side away from the operator. For example, a blade end of a knife used by a surgeon to create an incision in a patient's body is the distal end, while a handle end held by the doctor would be the proximal end.

As used herein, the term “endothelialized” refers to development and growth of endothelial tissue.

As used herein, the term “cardiovascular context” refers to understanding the various cardiovascular stressors placed on the cardiovascular system at any given point of time.

As used herein, the term “extracorporeal” refers to a thing that is anatomically outside the body.

As used herein, the term “health care professional” refers to at least any one or more of physician including but not limited to surgeon, first surgical assistant, physician's assistant, nurse practitioner, medical resident, medical student, nurse, nurse student, or any combination thereof.

While the present teachings are described in conjunction with various embodiments, it is not intended that the present teachings be limited to such various embodiments. On the contrary, the present teachings encompass various alternatives, modifications, and equivalents, as will be appreciated by those of skill in the art.

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein may be implemented with respect to any other method or composition described herein and that different embodiments may be combined.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

Throughout this specification, unless the context requires otherwise, the words “comprise”, “comprises” and “comprising” will be understood to imply the inclusion of a stated step or clement or group of steps or elements but not the exclusion of any other step or element or group of steps or elements. Further, the words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements. Reference throughout this specification to “one embodiment,” “one such embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present embodiment. Thus, the appearances of the foregoing phrases in various places throughout this specification are not necessarily all referring to the same embodiment. Furthermore, the particular features, structures, or characteristics may be combined in any suitable manner in various embodiments.

For purposes of this disclosure, a computer system may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a computer system may be a personal computer (e.g., desktop or laptop), tablet computer, a two-in-one laptop/tablet computer, mobile device (e.g., personal digital assistant (PDA), smart phone, tablet computer, or smart watch), server (e.g., blade server or rack server), a network storage device, or any other suitable device and may vary in size, shape, performance, functionality, and price. The computer system may include random access memory (RAM), one or more processing resources such as a central processing unit (CPU) or hardware or software control logic, ROM, and/or other types of nonvolatile memory. Additional components of the computer system may include one or more disk drives, one or more network ports for communicating with external devices as well as various input and output (I/O) devices, such as a keyboard, a mouse, touchscreen and/or a video display. The information handling system may also include one or more virtual or physical buses operable to transmit communications between the various hardware and/or software components.

### DETAILED DESCRIPTION

Aspects of the disclosure include systems and/or methods for reducing post heart pump placement infections, reducing implantation trauma to the heart, reducing effects of heart pump motor failure, and/or allowing for quick heart pump motor and/or battery upgrades. For example, the heart pump may comprise multiple parts that are in wireless communication with each other. Such a system when implanted within a patient would eliminate the need for wires extending from inside the body, to outside the body. Eliminating such wires would remove a major potential source of infection. For another example, placement of the cannula of the heart pump within the heart via a catheter instead of a surgical incision would significantly reduce the damage to the heart. Also, placing the heart pump motor outside the body thereby enabling a timely non-surgical swap in the event of a motor failure, mitigates what would otherwise be a catastrophic event that may result in death. In a similar vein, if motor technology or even battery technology improves, the motor and/or battery may be upgraded without the need for surgical intervention.

Other aspects of the disclosure systems and/or methods may include replacing the one or both sides of the heart ejection functionality. In one example, aspects of the heart pump may be placed into the left ventricle of a patient thereby replacing the left ventricular ejection function of the heart. Aspects of the heart pump may be positioned within the right ventricle of the patient, which would replace the right ventricular ejection function in another example. Also, in one embodiment, aspects of the heart pump may be placed in both the right and left ventricles of the heart and while working in tandem may replace both the right and left ventricular ejection function of the heart.

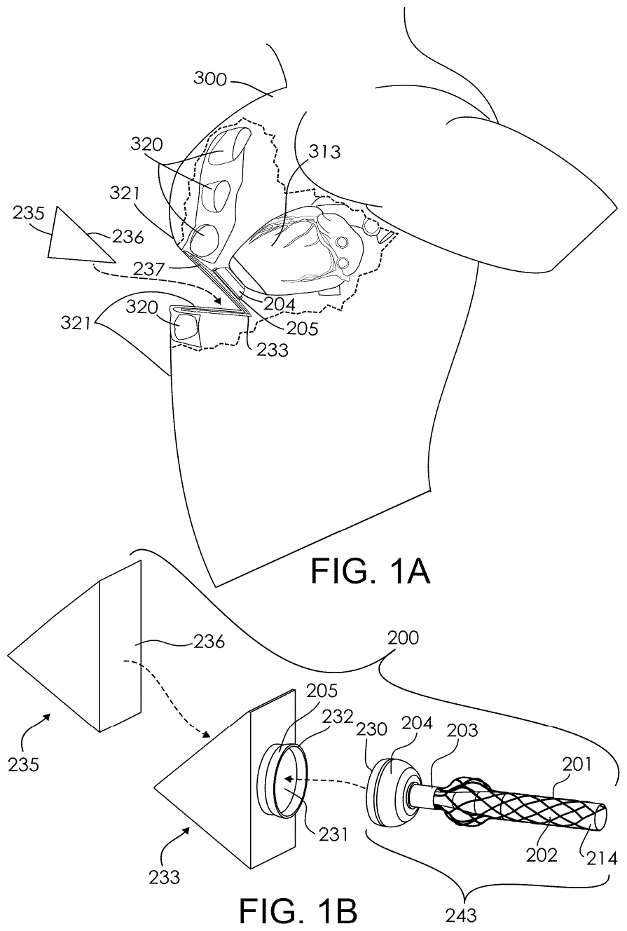

FIG. 1A and FIG. 1B illustrate an implantable heart pump device. Referring to FIG. 1A, the geometric housing may be secured to the outside of the skin of the patient but within a surgically created cavity, while the geometric plate may be implanted within the patient according to some embodiments of the disclosure. FIG. 1A further illustrates the attachment of the proximal end of the cannula to the geometric plate, and insertion of the distal end of the cannula into the heart of the patient. FIG. 1B illustrates how the geometric housing, geometric plate, and the cannula may piece together according to some embodiments of the disclosure.

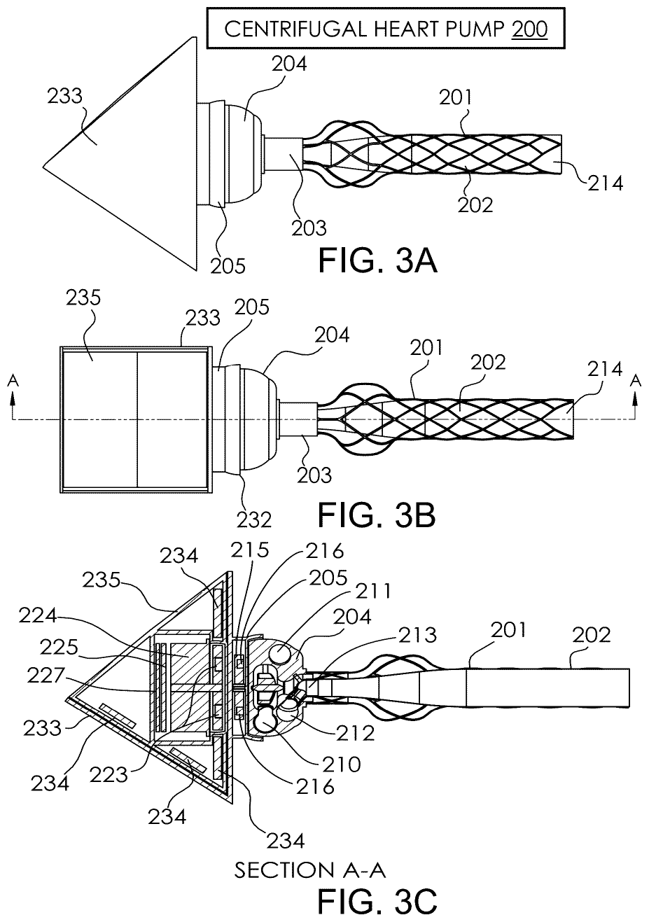

Referring to FIG. 1A, power may travel wirelessly from the geometric housing 235, which may be outside the body, to the impeller housing 204 implanted within the body, which in turn powers the cannula that may be inserted within the heart 313 thereby enabling normal cardiac flow. To this end, looking from outside the body of a patient 300 inward from a sideview perspective, the patient 300 includes a torso with a cutout, heart 313, ribs 320, and skin 321. The centrifugal heart pump 200 may be placed in the patient 300. For example, the geometric housing 235, which includes batteries as shown in FIG. 3C, may rest outside the body, but within a surgically created cavity. On one side of the geometric housing 235 there may be a geometric motor interface 236 which fits snuggly against the skin 321 of the patient 300 without penetrating the skin 321. Within the body of the patient 300, affixed snuggly to the interior aspect of the skin of the cavity may be the geometric plate 233. Coupled to the geometric plate 233 may be the body plate motor connector 237. The impeller housing 204 may then be coupled to the body plate motor connector 237 which may be connected to the cannula 243 of FIG. 1B that may be inserted within the heart 313.

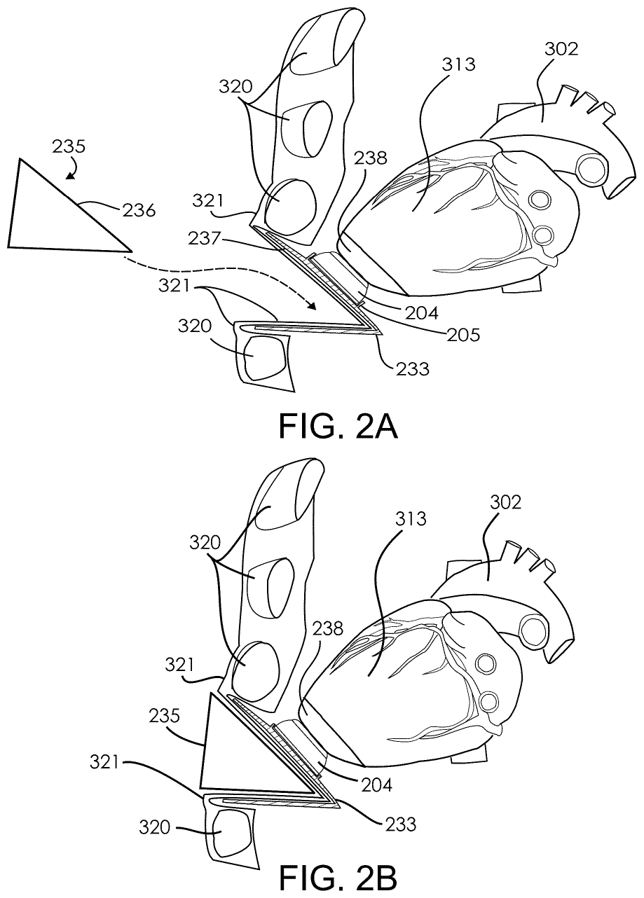

An example of a partially disassembled heart pump is illustrated in FIG. 1B. The heart pump 200 may be segmented into at least three parts: the dual cannula 243, the geometric plate 233, and the geometric housing 235. The dual cannula 243 may include an impeller housing ventricle interface 230, impeller housing 204, inflow conduit 203, outflow conduit 202, frame 201. The geometric plate 233 may include a ventricle magnetic driver housing 205, a ventricle magnetic impeller driver connector 232, and a ventricle magnetic impeller driver interface 231. The geometric housing 235 may have a geometric interface 236. In some embodiments, the geometric plate 233 and the geometric housing 235 may have the configuration of a triangle, circular, orthogonal, hexagonal, or any other different geometry. FIGS. 2A-B illustrate a side view example of how the geometric housing 235 may be secured within the cavity.

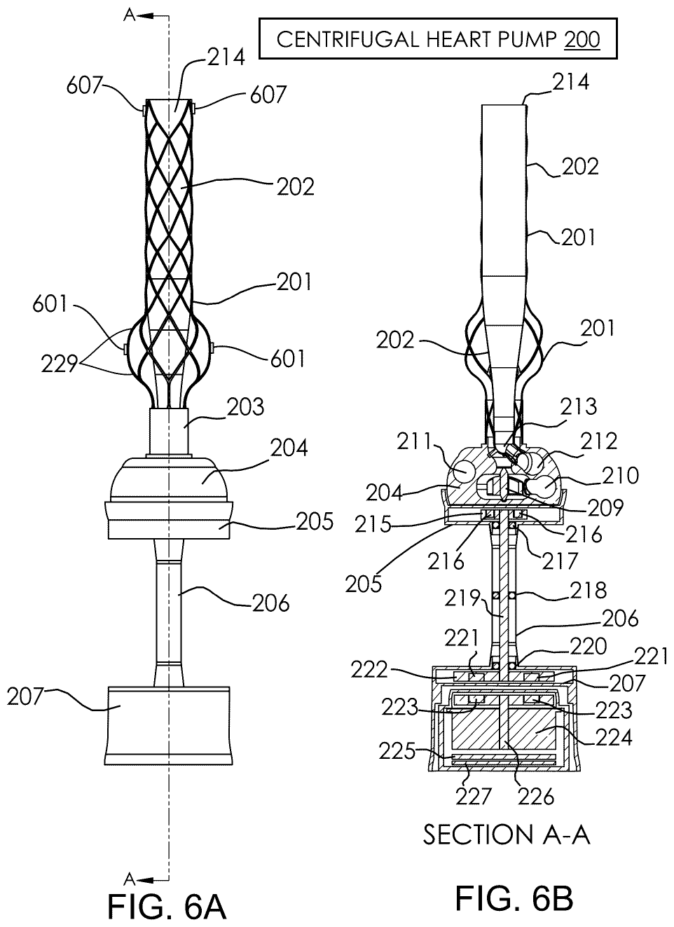

Turning now to FIGS. 3A-C, the centrifugal heart pump 200 has a frame 201 with an outflow conduit 202. The frame 201 may be made of a collapsible polymer, metal, or ceramic with varying dimensions. To this end, the frame 201 dimensions may include a wall thickness of 0.10 mm to 2 mm. The open dimension of the frame 201 may range from 8 mm to 25 mm, preferably 11 to 22 mm. However, the compressed diameter of the frame 201 may range from 4 to 15 mm, preferably 8 mm. The details of the geometric housing 235 and the impeller housing 204 are extensively covered in FIGS. 6A-6B.

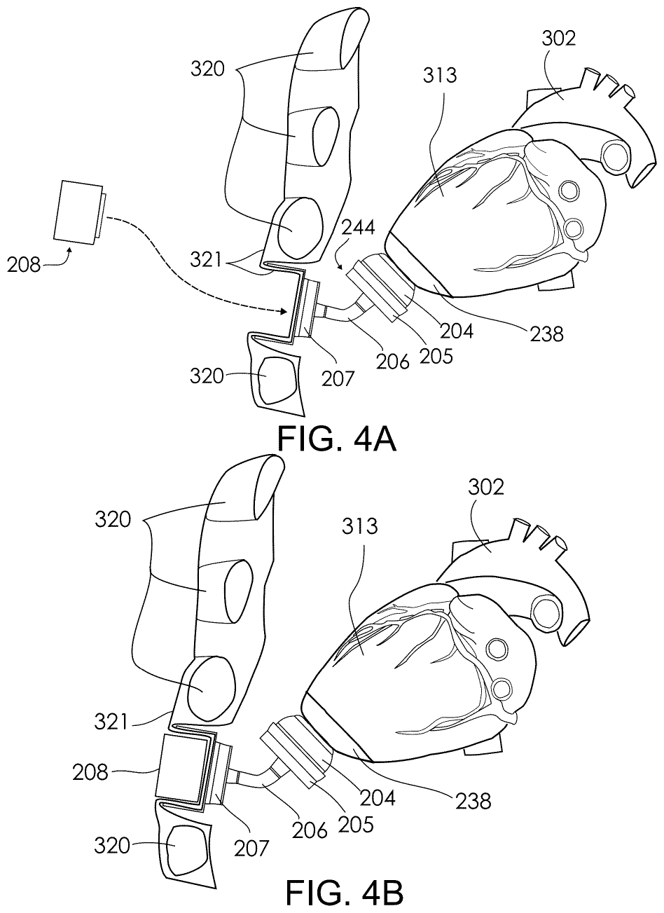

Referring now to FIGS. 4A-B, the geometric housing 208 (235 of FIGS. 1-3) moved from outside the patient cavity to within it thereby providing a wireless connection between the geometric housing 208 and the heart 313 via a flex drive 244. The geometric housing 208 may rest in the cavity secured in place by the magnets within the chest magnetic housing 207. In some examples, the geometric housing 208 may be a cube or a rectangle shaped. In between the geometric housing 208 and the chest magnetic housing 207 may be skin 321. The chest magnetic housing 207 of the flex drive 244 may have a shaft housing 206 that may be attached to the ventricle magnetic driver housing 205. The ventricle magnetic driver housing 205 may be attached to the impeller housing 204. The impeller housing 204 may be coupled to a heart mesh 238. The heart mesh 238 may be used to attach the impeller housing 204 to the heart 313 by means of suturing.

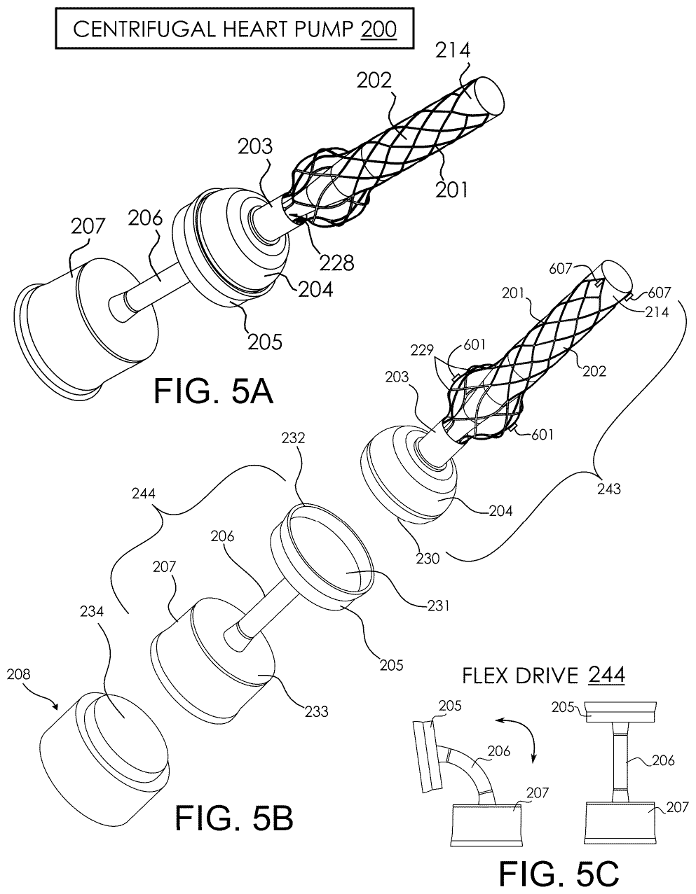

Looking at FIGS. 5A-5C, the flex drive 244 positioning within the centrifugal heart pump 200 is illustrated. In FIG. 5A, the flex drive 244 is shown attached to the chest magnetic housing 207 on proximal end and to the dual cannula 243 on the distal end. Referring to FIG. 5B, the centrifugal heart pump 200 may be divided into the motor housing 208, flex drive 244, and the dual cannula 243. In FIG. 5C, the flex drive 244 bends or flexes, which allows the centrifugal heart pump 200 to accommodate different anatomical configurations of patients 300.

FIGS. 6A-6B provides a side view and a cut away side view of the centrifugal heart pump 200 with the flex drive 244. Referring to FIG. 6A, the frame 201 has an inflow section 229. This may allow fluid to enter the inflow conduit 203, and in small or collapsed heart ventricles, the inflow section 229 may prevent obstruction to the inflow conduit 203. The frame 201 may have one or more inflow sensors 601. In one embodiment, the one or more inflow sensors 601 may at least measure pressure within the heart chamber, and heart ejection force, the oxygen levels of the blood, the pH level of the blood, and lactate level of the blood, etc. The pressure measurements may also include inflow pressure and ventricular forces. The ventricular forces may provide information on the recovery of the heart. Long-term use of a ventricular assist device may improve ventricular function. The force sensor may be used to measure the recovery of the ventricle if it occurs in the patient. Additionally, the one or more outflow sensors 607 are used to measure pressure within the heart chamber, and heart ejection force, the oxygen levels of the blood, the pH level of the blood, and lactate level of the blood, etc. One or more outflow sensor may be placed on the frame 201 near the one or more valves 214, or the outflow conduit 202 within the conduit. These sensors 601, 607 may be powered by radio frequency (RF) and transmit data through RF.

FIG. 6B illustrates a segmented view from FIG. 6A between section A-A cutting down the impeller housing 204, the ventricle magnetic driver housing 205, the shaft housing 206, and the chest magnet housing 207 which is covering the geometric housing 208. Looking first at the impeller housing 204, it may be cradled by the ventricle magnetic driver housing 205. The impeller housing 204 accommodates a centrifugal impeller 209. Next, the ventricle magnetic driver housing 205 contains a ventricle driver 215. For example, the ventricle driver 215 may drive the centrifugal impeller 209, which creates fluid flow. For another example, the ventricle driver 215 may contain the impeller driver magnets 216 that may drive the centrifugal impeller 209. Further, the shaft housing 206 contains the shaft 219 which may be connected to ventricle driver 215. The shaft 219 may be connected or attached to the chest magnet housing 222 in one embodiment. The shaft 219 may be maintained in place using one or more stabilizers 217, 218, 220. For example, the one or more stabilizers 217, 218, 220 consist of either bearings or magnets that are used to stabilize the shaft 219, minimizing vibrations. Additionally, the chest magnet housing 207 may contain the chest magnets. The chest magnet housing 207 may be coupled to the geometric housing 208. Moreover, the geometric housing clement 208 may contain an extracorporeal motor 224, which may have a motor shaft 226 that may be attached to motor magnet housing 245. The motor magnet housing 245 may contain motor magnets 223. The motor magnets 223 may by coupled to without contacting the chest magnet housing 222 by means of magnetic forces. The magnets do not require a physical connection. This allows the geometric housing 208, the flex drive 244, and the dual cannula 243 to be independent.

The geometric housing 208 may also contain a microprocessor 225 and battery 227. For example, the microprocessor 225 may drive the extracorporeal motor 224, communicate to an external device to provide data, and wirelessly charge the battery 227. In some embodiments, the battery 227 may power the extracorporeal motor 224 and microprocessor 225. The battery 227 may consist of one or more batteries allowing the patient 300 or health care professional to exchange a battery without the heart pump losing power. In another example, the heart pump may also be charged using a wire to the battery 227 by means of a cable such as but not limited to universal serial bus (USB), USB type A, USB type B, USB type C, USB 3.0, USB Mini, USB Micro A, USB Micro B, lightening, thunderbolt, etc.

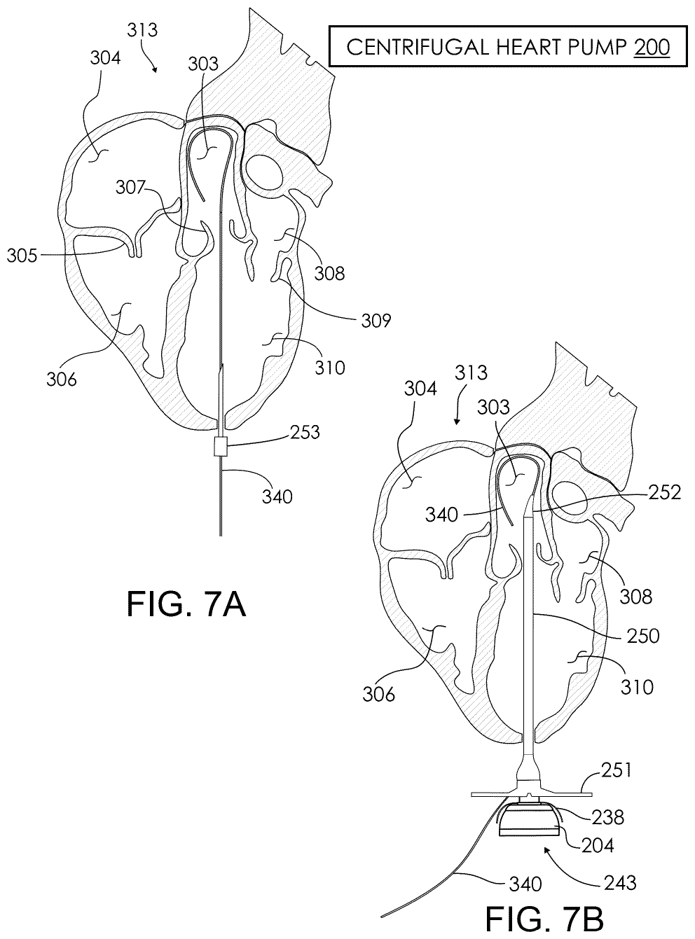

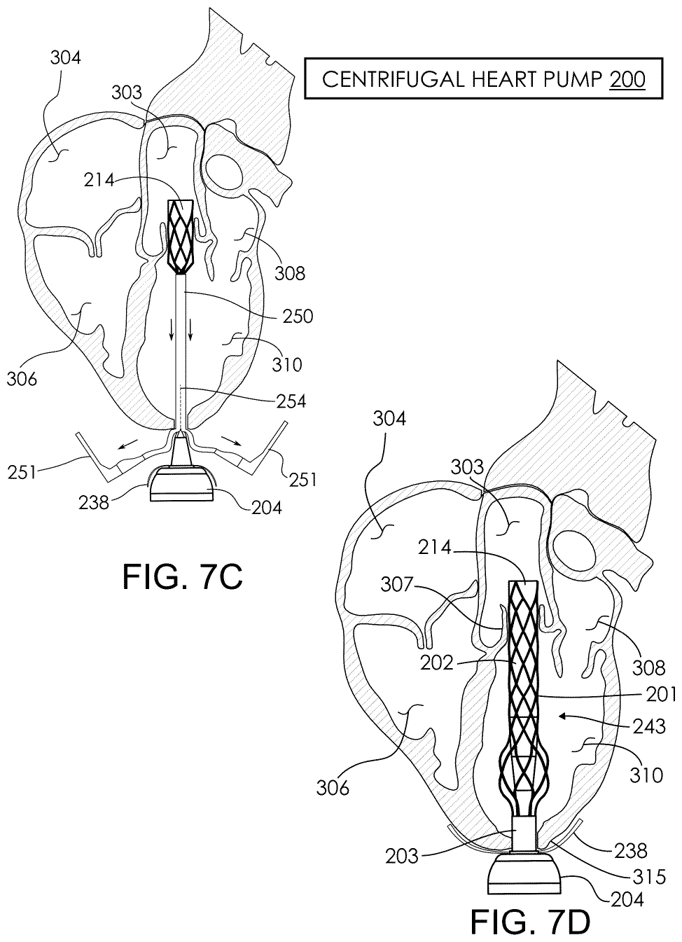

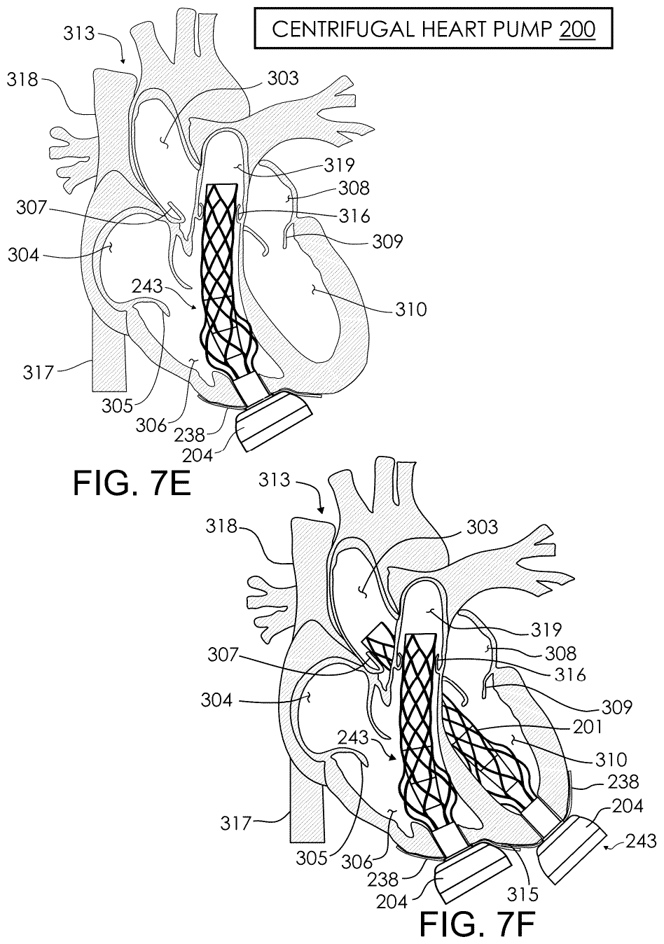

FIGS. 7A-7D shows one example of the insertion and deployment method of the dual cannula 243 into the heart 313. The heart 313 has several note worth points of anatomy to be aware of during such an insertion, including ascending aorta 303, right atrium 304, tricuspid valve 305, right ventricle 306, aortic valve 307, left atrium 308, mitral valve 309, pulmonary valve 316, inferior vena cava 317, superior vena cava 318, and pulmonary artery 319 are annotated. In this example, the left ventricle 310 of the heart 313 may be punctured using a needle 253. The guidewire 340 may be advanced through the needle into the left ventricle 310 on to the ascending aorta 303 as illustrated in FIG. 7A. The needle 253 may be removed from the guidewire 340. The dual cannula 243 may be collapsed within a sheath 250 as shown in FIG. 7B and may have an introducer tip 252 at the distal end. The dual cannula 243 within the sheath 250 may be advanced on the guidewire 340 thereby crossing the aortic valve 307 and moving into the ascending aorta 303 as illustrated in FIG. 7B. The sheath 250 may have a sheath handle 251, which allows health care professional to easily remove the sheath. The health care professional may begin to unsheathe or pull back the sheath 250 by using the sheath handle 251. Now referring to FIG. 7C, this process may allow the frame 201 and/or an outflow conduit 202 to self-expand and engage the aortic valve 307. The sheath 250 may have a peel away line 254 which may be thinner or have pre-perforated line to facilitate removal. If and when the sheath 250 is removed the remaining sections of the frame 201 may self-expand as shown in FIG. 7D. However, the impeller housing 204 may remain outside the heart 313. The dual cannula 243 may have a heart mesh 238 that may be used to fix the impeller housing 204 in place with sutures.

In another example, referring to FIG. 7E, the dual cannula 243 may be placed on the right side of the heart 313, possibly in the right ventricle 306. The introduction of the dual cannula 243 to the right side of the heart 313 may be similar to the left side process. In this example, the dual cannula 243 may be introduced to the right ventricle 306 extending through the pulmonary valve 316. The dual cannula 243 may be secured in place by sutures to the heart apex 315 or any wall of the heart 313. Further, the dual cannula 243 may endothelialized improving biological response and longevity of the device.

Referring to FIG. 7F, one or more dual cannulas 243 may be placed on both the left and right side of the heart, thereby replacing full heart function. The two or more dual cannulas 243 may work in harmony to replace heart ejection function. Such an embodiment may use dual extracorporeal motors 224 with either one or more geometric housing(s) 235, with one or more geometric plate(s) 233, or with one or more geometric housing(s) 208 with one or more flex drive(s) 244.

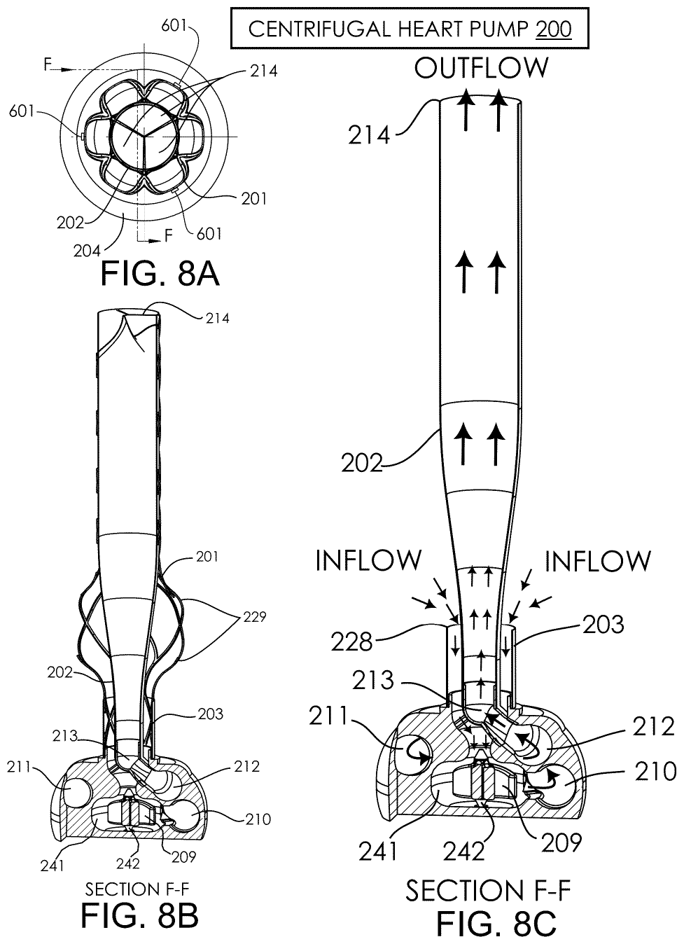

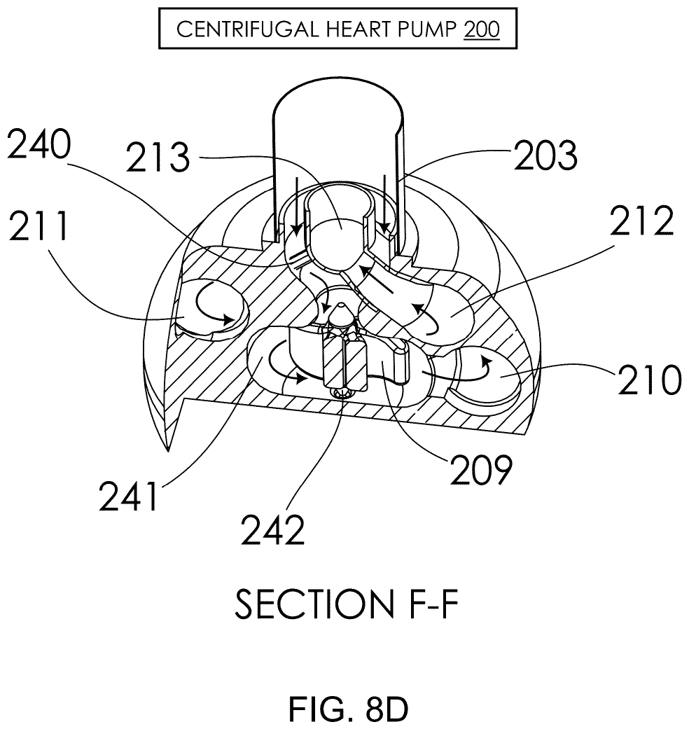

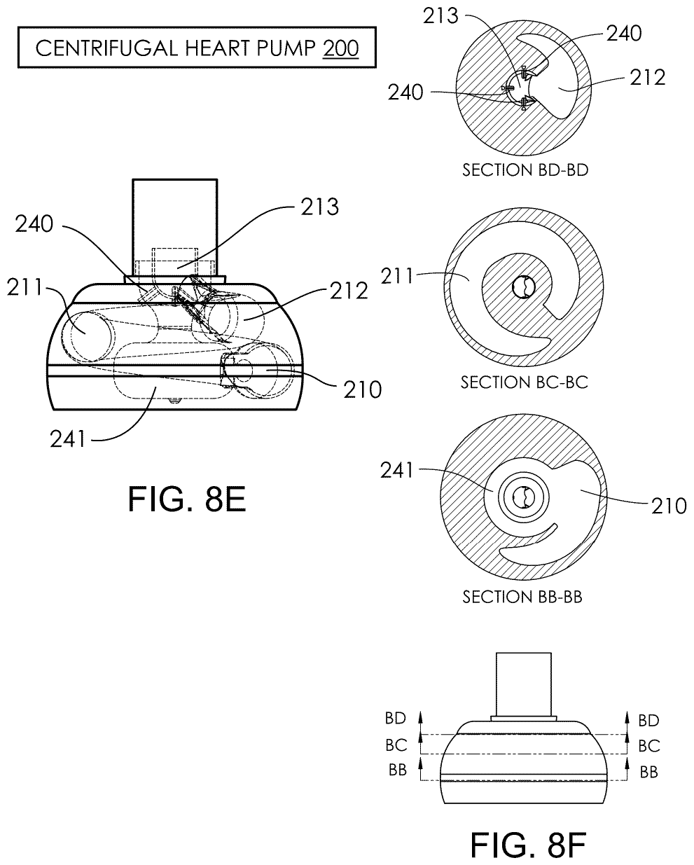

FIGS. 8A-8F illustrate a top down and cut a way view of the outflow conduit 202 and impeller housing 204. Turning to the FIG. 8A, the dual cannula 243 may be closed by one or more valves 214, and may contain one or more inflow sensors 601. FIG. 8B-D illustrates a partial side view and isometric view segmented from FIG. 8A between section F-F demonstrating one example of the dual cannula 243 flow profile through the spiral conduit. In this example, the fluid may enter the inflow conduit 203 between the inflow section 228 and the outflow conduit 202. The fluid may continue towards the volute 241 where it may meet the centrifugal impeller 209. The rotating centrifugal impeller 209 may be maintained in place by the impeller stabilizer 242. The centrifugal impeller 209 may rotate, generating centrifugal forces, as the name suggests, which may then propel the flow radially into channel-one 210. The propelled fluid may continue through channel-two 211, past channel-three 212, upward to channel-four 213 exiting through the outflow conduit 202 and through the one or more valves 214 of the dual cannula 243. FIG. 8C denotes an example of the inflow and outflow of the dual cannula 243 without the frame 201 in order to better appreciate the flow profile. FIG. 8D illustrates a segmented zoomed in view of the flow from FIG. 8A without the frame 201 and outflow conduit 202. Of note, the junctions 240 are spaced equally allowing flow to enter the volute 241. FIGS. 8E-8F show top down and side view of the spiral conduit. FIG. 8E illustrates a side view of the spiral conduit within the impeller housing 204 as indicated by the dash line. The spiral conduit may be further appreciated in FIG. 8F which illustrates top view of three sections: BB-BB, BC-BC, and BD-BD.

Turning from the centrifugal heart pump 200 embodiments discussed above, another example of a heart pump may include an axial heart pump 106. An axial heart pump 106 may comprise either a geometric housing 235 and geometric plate 233, or a geometric housing 208 with a flex drive 244.

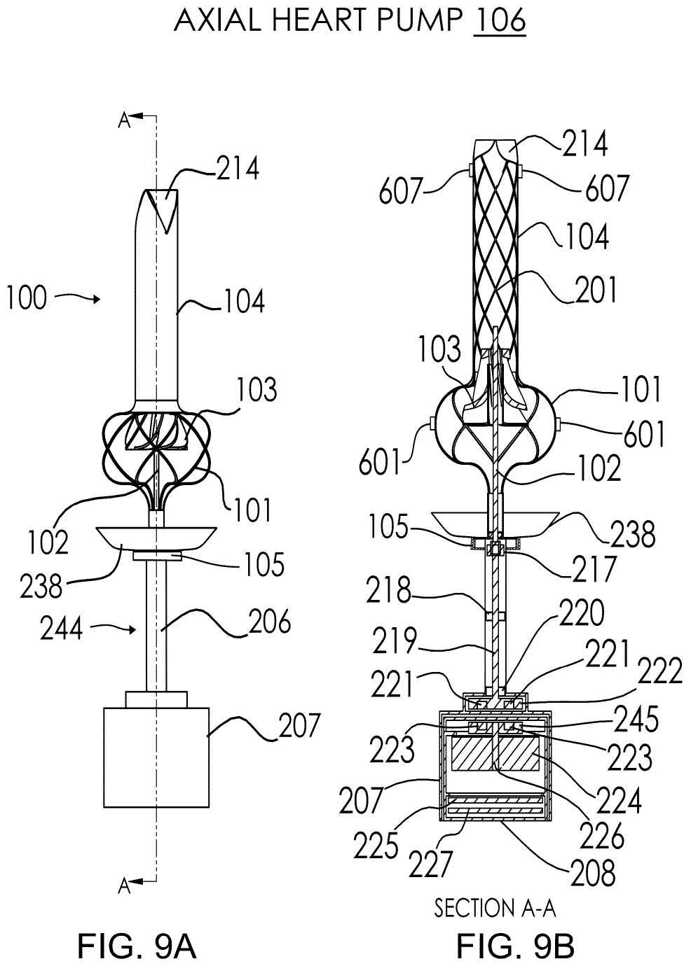

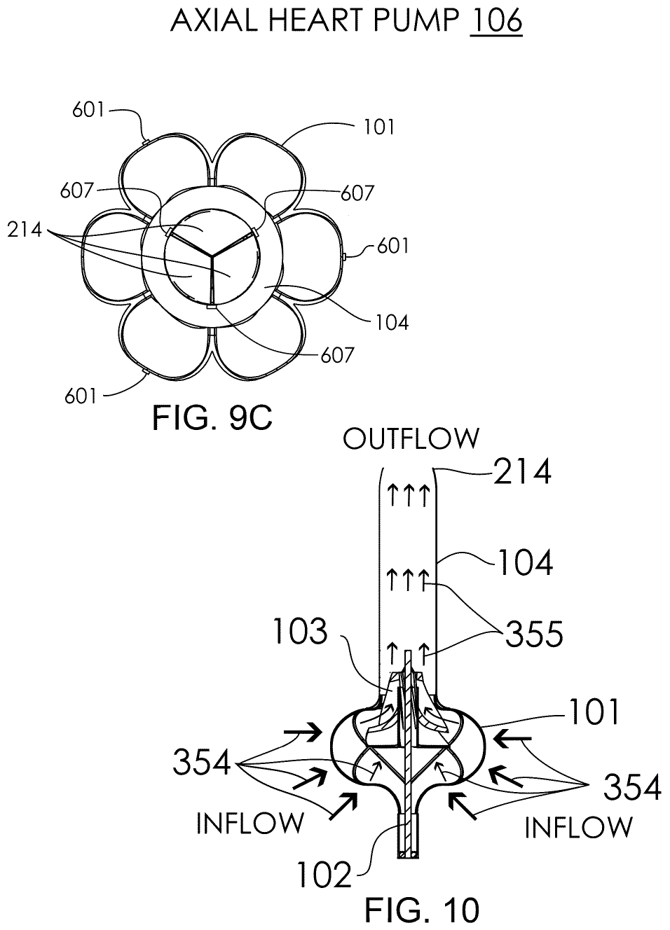

FIGS. 9A-C illustrates a top down, side, and side cross-cut view of one example of an axial heart pump 106. Referring to FIG. 9A, the axial heart pump 106 may be coupled to an axial cannula 100. The axial cannula 100 consists of an axial conduit 104, frame 201, shield 101, axial shaft 102, axial impeller 103, heart mesh 238. Further, the axial conduit 104 may have one or more valves 214. The one or more valves 214 may open and close as the pressure around the valves change. The one or more valves 214 opens when the pressure inside the axial conduit 104 may be greater than the pressure outside the one or more valves 214. When the pressure may be greater outside the one or more valves 214, the one or more valves 214 may close preventing valvular regurgitant flow, a life threatening situation. The one or more valves 214 will continue native valve function depending on patient ventricular pulsatility or until the health care provider enables the extracorporeal motor 224 and pump function begins. In one example, the axial impeller 103 may be collapsible. The axial impeller 103 may be collapsed by means of folding the blades or vanes. The axial impeller 103 may attach to and be driven by the axial shaft 102. The axial shaft 102 may be made up of a polymer, metal alloy, or combination.

Turning now to FIG. 9B, the flex drive 244 is connected to the axial conduit 104 with a frame 201. The section of the frame 201 that may not be covered by the axial conduit 104 may be covered by the shield 101. The shield 101 may be of a larger diameter than the axial conduit 104 and may partially interact with the ventricle to be able to measure forces and reduce obstruction of flow.

Continuing to reference FIG. 9B, the flex drive 244 may not have the ventricle magnetic driver housing 205 as shown in some embodiments. For example, the cannula 100 may be connected to the flex drive 244 by means of the connector 105 instead. The connector 105 may attach to the cannula 100 to drive the axial shaft 102. The axial shaft 102 may be in communication with the shaft 219 of the flex drive 244. The shaft 219 of the flex drive 244 may be coupled with one or more stabilizers 217, 218, 220 that provide stability and minimize vibration during operation. The one or more stabilizers 217, 218, 220 as well as the shaft 219 of the flex drive 244, may be located in the shaft housing 206. In one example, the flex drive 244 may have a circular magnetic housing 207 that may connect to the geometric housing 208. The shaft 219 may be connected to the chest magnet housing 222, which may house the chest magnets 221. These chest magnets 221 may be used to interact with the motor magnets 223 which may be housed inside the geometric housing 208. The chest magnets 221 and motor magnets 223 may consist of one or a plurality of magnets. The motor magnets 223 may be attached to the motor shaft 226, which may be driven by the extracorporeal motor 224. In another example, the geometric housing 208 may encompass the microprocessor 225 and battery 227. The microprocessor 225 and battery 227 may operate the extracorporeal motor 224. The battery 227 and microprocessor 225 may collect data from the one or more inflow sensors 601 and one or more outflow sensors 607.

Referring to FIG. 9C, the axial cannula 100 may be scaled off by one or more valves 214. One or more inflow sensors 601 and one or more outflow sensors 607 may be coupled or in proximity to the one or more valves 214.

FIG. 10 illustrates the axial cannula flow profile. As the axial impeller 103 rotates it may create a suction force from outside of the shield 101. The flow 354 from outside the cannula may begin to be propelled by axial impeller 103 through the inside of the axial conduit 104. The flow 355 may advance towards the one or more valves 214 to the outflow. The axial cannula 100 may be endothelialized improving biological response and longevity of the device.

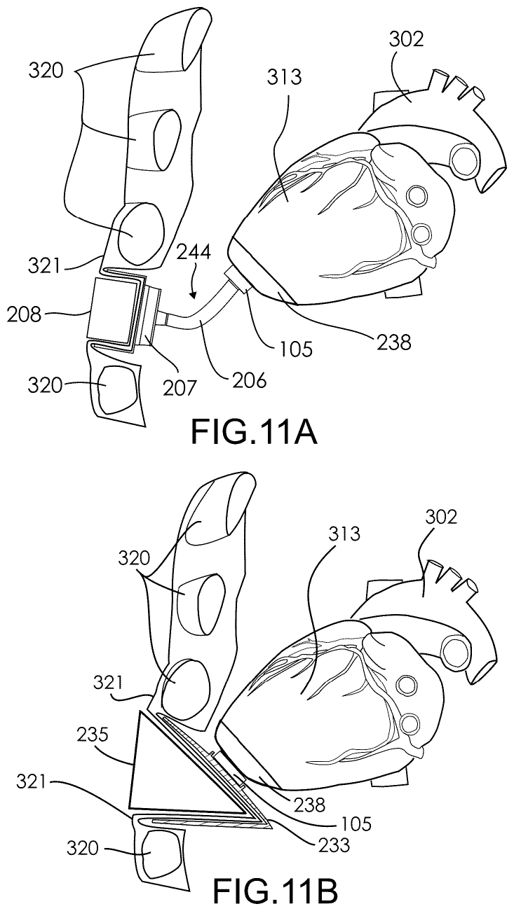

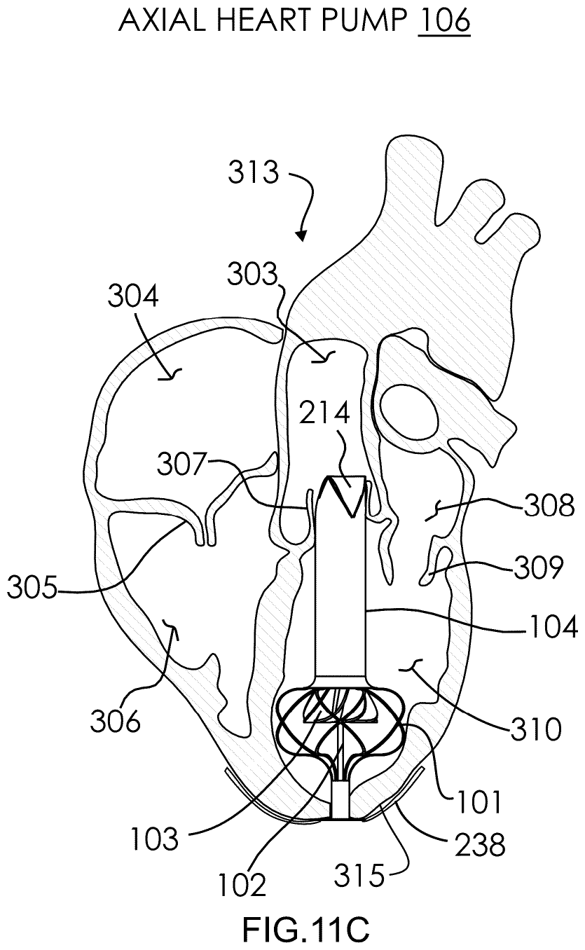

Looking at FIGS. 11A-11B, the axial heart pump 106 may be coupled to the flex drive 244 or geometric plate 233 the same way as the centrifugal heart pump 200 was as seen in FIGS. 2A-2B.

Referring to FIG. 11C, the axial cannula 100 may be place inside the heart 313 via the same insertion process as the dual cannula 243 as seen in FIGS. 7A-D. In one example, the axial cannula is placed in the right ventricle 310 and extends through the ascending aorta 303. The shield may push against the nearby heart tissue enabling the axial impeller 103 to freely rotate. In another example, the axial cannula 100 may similarly be placed in the right ventricle 306 or both ventricles 306 310 at the same time.

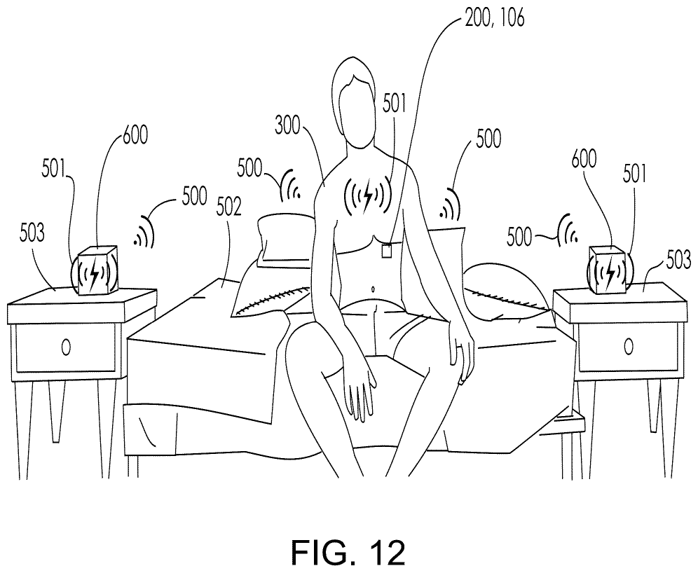

Referring to FIG. 12, the patient 300 may have the ability to communicate with the heart pump 200, 106 by means of a communication device 600. For example, the patient 300 may be in a bed 502 and the communication device 600 may be on top of a nightstand 503. The communication device 600 may have a wireless connection 500 to the heart pump and the heart pump batteries may enjoy wireless charging 501.

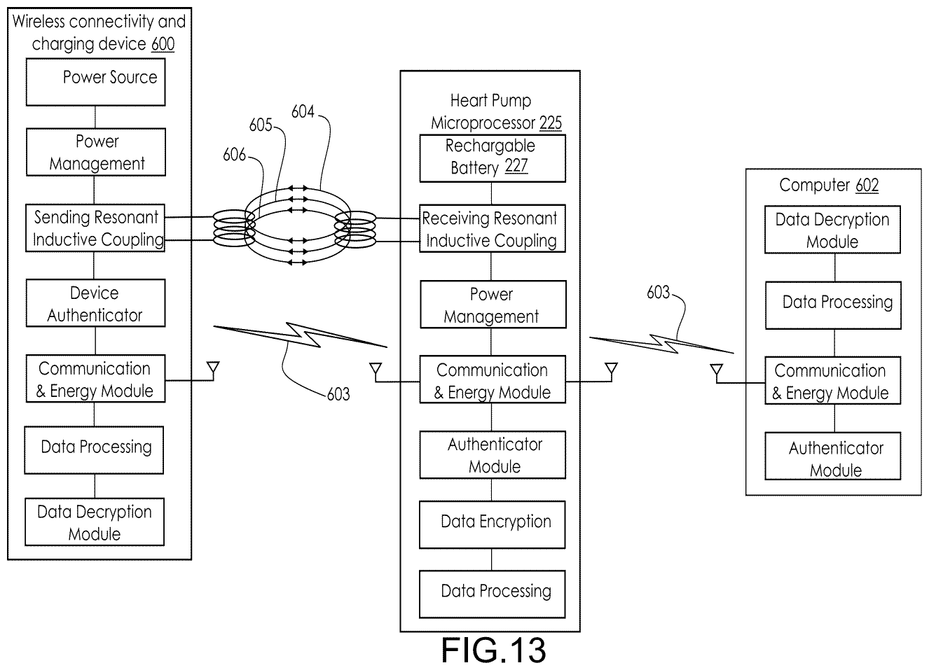

FIG. 13 is a block diagram of the wireless connectivity and charging device 600, the heart pump microprocessor 225, and the computer 602. In another example, the heart pump 200 106 may have a microprocessor 225 that may have the ability to operate the extracorporeal motor 224, communicate to an external device such as a communication device 600 or computer 602 (FIG. 12-13). The computer may consist of a tablet or phone. The microprocessor 225 may have an authenticator module, data encryption, data decryption, power management, data processing abilities. For example, the computer system network interfaces may include, but are not limited to Cellar, Satellite, Wide Area Network, Fiber, Broadband, Cable, DSL, RF 603 such as Wireless Fidelity (Wi-Fi) at frequencies of 2.4 GHz and 5 GHz, or Bluetooth (2.4 GHz ISM band). In some embodiments, an auxiliary wired electrical cable may be used for charging and communication. The wireless charging may be resonant inductive coupling 604, 605, 606. Power transmission to the rechargeable battery 227 of FIG. 6B may also be in the form of radio frequency (RF) 603. Further, RF 603 may be used for communication as well.

One embodiment may include, a heart pump system comprising a cannula comprising a ventricle interface coupled to an impeller housing, where the impeller housing is coupled to a conduit, and the impeller housing comprising an impeller. One example may include a cannula 100 that comprises a ventricle interface 230 that is connected to the impeller housing 204 which is in turn connected to a conduit. For another example, the impeller housing may include a centrifugal impeller 209 or an axial impeller 103. Another embodiment may include, the ventricle interface comprising impeller driver magnets, where the impeller driver magnets are configured to rotate the centrifugal impeller or the axial impeller in the presence of a magnetic field. For example, the ventricle interface 230 may comprise impeller driver magnets 216 which may rotate centrifugal impeller 209 or the axial impeller 103 in a presence of a magnetic field. In some embodiments, a motor housing comprising a motor and motor magnets, with the motor coupled to the motor magnets and configured to rotate the motor magnets, wherein the motor housing is configured to generate the magnetic field that rotates the impeller. In one example, a motor housing 208 may comprise a motor 224 configured to rotate the motor magnets 223 creating a magnetic field that may then rotate the centrifugal impeller 209 or axial impeller 103.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

What is claimed is:

1. A heart pump system, comprising:
a cannula comprising a ventricle interface coupled to an impeller housing, the impeller housing coupled to a conduit, the impeller housing comprising an impeller, the ventricle interface comprising impeller driver magnets, the impeller driver magnets configured to rotate the impeller in a presence of a magnetic field; and
a motor housing comprising a motor and motor magnets, the motor coupled to the motor magnets and configured to rotate the motor magnets, wherein the motor housing is configured to generate the magnetic field that rotates the impeller.

2. The heart pump system of claim 1, wherein the impeller comprises a centrifugal impeller or an axial impeller.

3. The heart pump system of claim 1, wherein the motor housing is configured to rotate the impeller with the motor without contacting the ventricle interface.

4. The heart pump system of claim 1, wherein the motor housing comprises a battery coupled to the motor, and wherein energy stored in the battery is used to rotate the impeller.

5. The heart pump system of claim 1, further comprising:
a plate comprising a first side and a second side with an impeller driver connector on the second side, the impeller driver connector configured to align the impeller driver magnets of the ventricle interface with the motor magnets of the motor housing when the motor housing is placed alongside the first side.

6. The heart pump system of claim 1, wherein the conduit comprises a spiral conduit.

7. The heart pump system of claim 1, wherein the ventricle interface is coupled to the cannula through a flexible shaft.

8. The heart pump system of claim 6, wherein the cannula comprises: a centrifugal impeller; and a stabilizer coupled to the centrifugal impeller.

9. The heart pump system of claim 1, wherein the cannula comprises a self-expandable cannula.

10. The heart pump system of claim 1, wherein the conduit comprises a valve at an end of the conduit opposite the impeller.

11. The heart pump system of claim 1, wherein the motor housing is positioned extracorporeally and replaced or removed.

12. The heart pump system of claim 1, wherein the cannula comprises one or more of a plurality of physiological sensors.

13. The heart pump system of claim 12, wherein the motor housing comprises a processor configured to perform steps comprising:
receiving, from the physiological sensor, a physiological measurement value;
determining, by the processor, whether the physiological measurement value is below a threshold; and
adjusting, by the processor, a speed of the motor based on determining by an artificial intelligence algorithm that the physiological measurement value is below the threshold.

14. The heart pump system of claim 13, wherein the processor is configured to perform steps comprising:
receiving, from the physiological sensor, a first physiological measurement value;
receiving, from the physiological sensor, a second physiological measurement value;
determining, by the processor, a historical value based on the first physiological measurement value and the second physiological measurement value; and
determining, by the processor, the threshold based on the historical value.

15. An apparatus, comprising:
a cannula comprising a ventricle interface coupled to an impeller housing, the impeller housing coupled to a conduit, the impeller housing comprising an impeller coupled to an impeller,
the ventricle interface comprising impeller driver magnets, the impeller driver magnets configured to rotate the impeller in a presence of a magnetic field.

16. The apparatus of claim 15, further comprising a flexible shaft coupled to the ventricle interface.

17. The apparatus of claim 15, wherein the cannula comprises a stabilizer coupled to the impeller.

18. The apparatus of claim 15, wherein the ventricle interface is coupled to the cannula through a flexible shaft, and the cannula comprises: a centrifugal impeller; and a stabilizer coupled to the centrifugal impeller.

19. A method, comprising:
accessing a chest cavity of an individual by surgical incision of a chest wall;
implanting a cannula within a heart through the chest cavity, the cannula comprising a ventricle interface coupled to an impeller housing, the impeller housing comprising an impeller coupled to impeller driver magnets; and
coupling a motor housing outside of the individual to the ventricle interface of the cannula for rotating the impeller by application of a magnetic field that rotates the impeller driver magnets.

20. The method of claim 19, wherein the cannula is advanced within the heart through a target cardiac valve.
